(19) United States
(12) Patent Application Publication
(10) Pub. No.: US 2011/0245790 A1
(43) Pub. Date: Oct. 6, 2011

(54) NIGHT SWEAT PAD

(76) Inventor: Richard Earl Castro, Waikoloa, HI (US)

(21) Appl. No.: 12/752,029

(22) Filed: Mar. 31, 2010

(51) Int. Cl. A61F 13/15

(52) U.S. Cl. 604/367

(57) ABSTRACT

A sweat pad designed to be placed on the bed and under a user for wick away the moisture from a user is disclosed. The sweat pad comprises a moisture-absorbent filler sandwiched between a top moisture-permeable sheet and a bottom cloth sheet that is not moisture proof from the outside. The top moisture-permeable sheet is configured to wick moisture away from the user and toward the moisture absorbent filler, thus giving the user a dry top layer to sleep on in comfort.

### FIELD OF THE INVENTION

This invention generally relates to a sweat pad and more particularly to a sweat pad that wicks moisture away from the human body.

### BACKGROUND OF THE INVENTION

Humans frequently experience night sweats while sleeping. Moisture from night sweats can cause discomfort that interferes with sleep. In addition, moisture from night sweats can penetrate through a lower bed sheet and damage the bed mattress or other bedding. Various bed coverings have been described in the prior art for preventing water and other forms of moisture from penetrating through a lower bedsheet and damaging the bed mattress or other bedding. Generally, these bed coverings comprise a water-resistant covering that is placed on the bed beneath the user. As used herein, the term “water-resistant” is intended to include materials that completely prevent water penetration, as well as materials that allow a small amount of moisture to penetrate under some conditions. The water-resistant covering may be directly in contact with the user, or covered by a water-resistant sheet. This water-resistant sheet can be uncomfortable when directly in contact with the user. While, the discomfort is reduced by covering the water-resistant sheet with a water-permeable covering, there is the necessity to remove the water-permeable covering when it is soiled.

U.S. Pat. No. 5,701,617 discloses an easily changeable, moisture resistant bed sheet that is designed to be placed under the user. The absorbent sheet includes water-permeable upper and lower textile sections, a moisture-resistant center section, a moisture absorbent pad on the center section, and a textile cover sheet component of the same material as the upper and lower sections of the bottom sheet component releasably secured over the pad and center section to give the appearance of a conventional single sheet formed of a single textile fabric.

U.S. Pat. No. 4,372,309 discloses a moisture absorbent pad designed for absorbing urine and useful in incontinence pads, bed pads, or disposable diapers designed for use by infants. The moisture absorbent pad includes a diffuser layer interposed between layers of moisture absorbent material situated within a cover sheet. The diffuser layer comprises one or more tissue layers, each having a plurality of parallel corrugations to disperse moisture by capillary action. A compression seal is situated in the cover sheet separating two pad parts. The compression seal comprises first and second crimps, spaced by a section of the cover sheet. The ends of the pad parts are also sealed by crimping. The absorbent material is wood pulp impregnated with a starch polymer or an acrylic-based polymer to increase the absorbency thereof. An agent may be included in the pulp to prevent the biological degradation of urine. Substances may also be included in the pulp for absorbing volatile nitrogenous compounds and odors. In addition, a moisture or chemical sensitive indicator strip may be situated on the cover sheet.

U.S. Pat. No. 6,223,076 discloses a sweat control or hyperhidrosis treatment device for providing iontophoresis of antiperspirant into a region of a human body, which includes a DC power source, a controller and a pair of electrodes. The electrodes are mounted in generally close proximity to one another and are separated by an insulating member. The electrodes generally carry an antiperspirant element and are responsive to a current signal through the controller. The controller converts the DC signal to an AC waveform. The device further includes a pair of moisture absorbing pads. Each of the pads is positioned in adjacent contact with one of the electrodes and preferably carries sodium salicylate to increase the permeability of the region. The electrodes are sized and arranged so that the tissue to be treated can extend across the insulating member and simultaneously contact both of the pads. The pads generally carry an aluminum-based antiperspirant such as aluminum-chlorohydrate or aluminum-zirconium or may include an anticholinergic substance.

U.S. Pat. No. 6,603,054 discloses a fibrous absorbent structure that is wet stable and has large void volume with a density below the critical density of the fiber employed. In one embodiment, the fibrous absorbent uses open-celled foam technologies to keep the fibrous structure expanded and bonded. In other embodiments, the resulting fibrous structure resembles an open-celled polymeric foam, with fibers serving as struts stabilized by binder material. In another embodiment, the resulting fibrous structure is filled with hydrophilic open-celled foams with the cell size substantially smaller than the fibrous pores. Such a wet-stable, high void volume fibrous absorbent can be used in a disposable product intended for the absorption of fluid such as body fluid, including extensible absorbent articles.

U.S. Pat. No. 6,551,297 discloses an absorbent article includes a liquid-permeable top layer, a liquid-impermeable back layer, and a liquid-retentive absorbent member interposed between the top layer and the back layer. The absorbent member can be absorbent paper, nonwoven fabric, a pulp sheet made of fibers and a binder, fluff pulp, a sheet obtained by interposing a superabsorbent polymer between paper or nonwoven fabrics or between paper and nonwoven fabric in an overlaid configuration or a sheet made of a mixture of a superabsorbent polymer and fiber or hydrophilic fiber, and the like. A sheet of a mixture of a superabsorbent polymer or hydrophilic fiber is preferred for its absorptivity for a body fluid (a sheet obtained by admixing a hydrophilic fiber, a superabsorbent polymer and a binder, and forming it into a sheet-like shape). The superabsorbent polymer in the sheet may be dispersed either in layers or in three dimensions.

U.S. Pat. No. 6,274,218 discloses a body fluids absorbent article including a liquid-pervious topsheet, a liquid-impervious backsheet and a liquid-absorbent core disposed between the topsheet and the backsheet. The topsheet includes a plurality of liquid-pervious apertures, an upper fibrous layer having a relatively low density and a lower fibrous layer having a relatively high density. Around each of the apertures the first and second fibrous layers are integrated together so that the topsheet has a density progressively increasing from an upper surface towards a lower surface of the topsheet and has, in the proximity of the lower surface, a density further higher than in the second fibrous layer. The first fibrous layer is formed by a web of thermoplastic fibers, preferably of crimped conjugated fibers mechanically entangled or heat-sealed together, or more preferably by a nonwoven fibrous sheet such as a nonwoven fabric. The second fibrous layer is also formed by a plurality of thermoplastic synthetic fibers mechanically entangled or heat-sealed together, more preferably provided in the form of a nonwoven fabric. The backsheet is made of a plastic sheet and the absorbent core is made of a mixture of fluff pulp fiber and superabsorptive polymer articles.

It is within this context that embodiments of the present invention arise.

### BRIEF DESCRIPTION OF THE DRAWINGS

Other objects and advantages of the invention will become apparent upon reading the following detailed description and upon reference to the accompanying drawings in which:

FIG. 1A is a top view illustrating use of a night sweat pad according to an embodiment of the present invention.

FIG. 1B is a side cross-sectional view illustrating a night sweat pad according to an alternative embodiment of the present invention.

FIG. 1C is a three-dimensional view illustrating a night sweat pad configured as a pillow case according to an alternative embodiment of the present invention.

FIG. 2 is an exploded perspective view of a night sweat pad according to a preferred embodiment of the present invention.

FIG. 3 is a partial cross-sectional view of the night sweat pad of the type depicted in FIG. 2.

### DESCRIPTION OF THE SPECIFIC EMBODIMENTS

Although the following detailed description contains many specific details for the purposes of illustration, anyone of ordinary skill in the art will appreciate that many variations and alterations to the following details are within the scope of the invention. Accordingly, the exemplary embodiments of the invention described below are set forth without any loss of generality to, and without imposing limitations upon, the claimed invention.

### INTRODUCTION

Moisture wicking fabrics have been developed that can wick moisture away from the skin preventing the moisture from being trapped between the fabric and the skin. Such fabrics have been used in pajamas, nightgowns and travel robes. Although such fabrics can keep the wearer dry by wicking moisture away, when the wearer is sleeping, the moisture can be undesirably wicked into the mattress or other bedding.

Moisture wicking fabrics have been used in multi-layer constructions for clothing such as outdoor apparel and also for footwear. In a typical construction, a filler material designed for warmth is sandwiched between a regular cloth inner layer and an outer layer of moisture wicking fabric. This type of construction is worn with a technical fabric layer closest to the skin and the moisture wicking layer furthest from the skin. However, the fabric of the outer layer is typically made of a waterproof material configured to prevent moisture (e.g., rain) from absorbed by the filler. Such a construction would not make an effective night sweat pad because the outer waterproof layer would not provide sufficient friction with a bedsheet to allow a user to sleep comfortably.

Prior art constructions used for liquid absorbing purposes, e.g., in sanitary napkins as described above with respect to U.S. Pat. No. 6,551,297 have an absorbent material between a liquid-permeable top sheet and a liquid-impermeable back sheet. If the liquid permeable layer were to wick moisture into the absorbent material, the moisture would be trapped in the absorbed material by the top sheet and back sheet. While this may be desirable for disposable incontinence pads and sanitary napkins it is undesirable if the construction is to be reused.

### EMBODIMENTS

Embodiments of the present invention are directed a composite absorbent sweat pad that can be used by people who frequently experience night sweats. In embodiments of the present invention, a night sweat pad includes a filler material sandwiched between a top layer of moisture wicking fabric and a bottom layer of regular cloth. The moisture wicking fabric is configured to wick moisture into the filler material. When the pad is placed on a bed with the moisture wicking fabric closest to the user's skin moisture from night sweats is wicked away from the user's skin into the filler layer and prevented from entering the bedding by the regular cloth outer layer. However, the use of regular cloth in the outer layer allows moisture from sweat to be removed from the filler material by conventional laundering and drying as opposed to being permanently trapped by a moisture-impermeable layer.

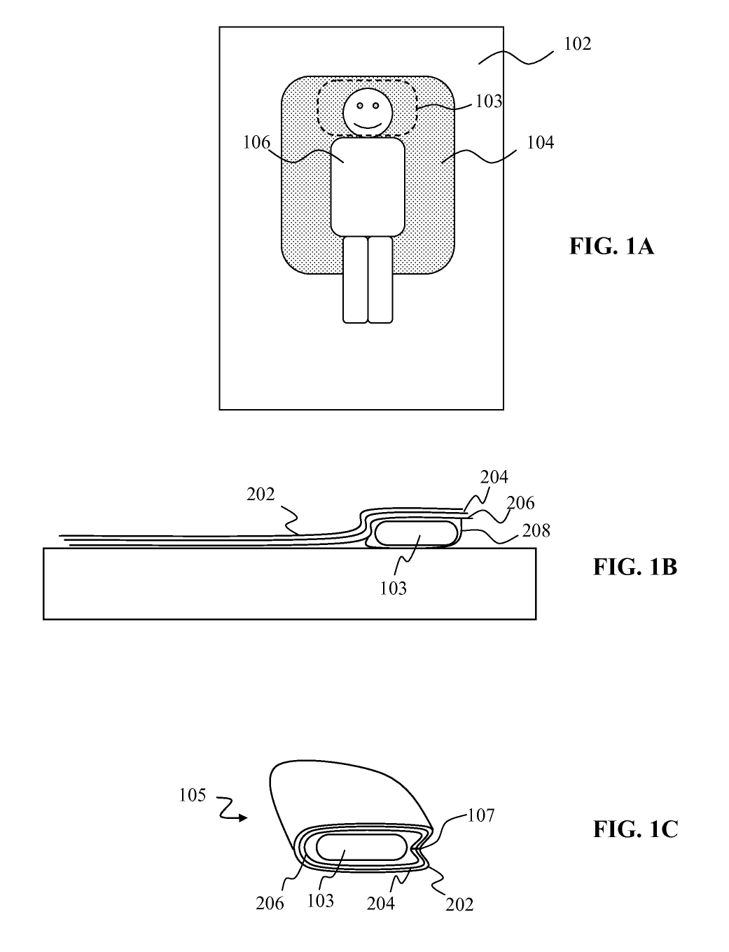

By way of example, and not by way of limitation, a sweat pad 104 can be placed on a bed 102 and/or on a pillow 103 and under the user 106 as shown in FIG. 1A. In general the sweat pad 104 is sufficiently long and sufficiently wide that a user can lie down on it while sleeping. By way of example, and not by way of limitation, the sweat pad 104 may have a length that is slightly shorter than a user of average height and width that is slightly narrower than a typical bed, e.g., a twin size bed. Larger or smaller sized dimensions may be used depending on the size of the user or the bed and/or pillow for which it is designed.

The sweat pad may have any suitable dimensions. By way of example, and not by way of limitation, the sweat pad may be characterized by a length between about 34 inches and about 75 inches and a width between about 21 inches and about 39 inches. Examples of embodiments of the present invention include versions for pillows that are about 21 inches by 34 inches. Other versions include a single pad measuring about 36 inches by about 48 inches. Yet other versions include a pad/pillow combination measuring about 36 inches by about 60 inches and a twin pad/pillow combination measuring about 39 inches by about 75 inches.

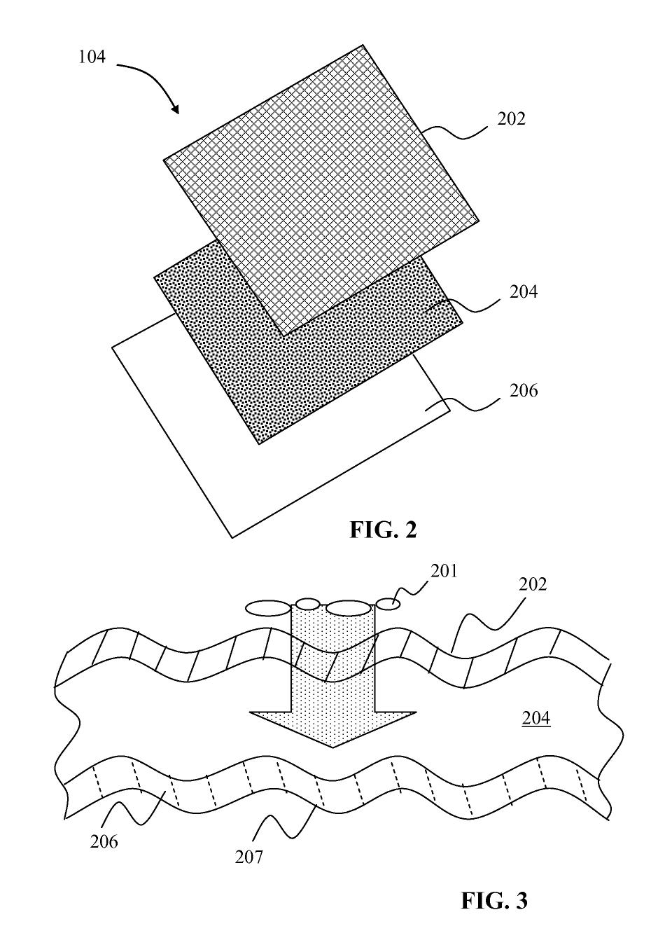

FIGS. 2 and 3 are an exploded perspective view and a cross-sectional view respectively of the sweat pad 104 of FIG. 1A. As shown in these figures, the sweat pad 104 comprises of a top sheet 202, a bottom sheet 206 and a moisture-absorbent filler sheet 204 sandwiched between the top sheet 202 and the bottom sheet 206. The top sheet 202, which is in contact with the user's body 106, is moisture-permeable and typically made of a technical fabric configured to wick sweat moisture away from the skin. The top sheet is made of a fabric that is moisture permeable in one direction. Specifically, the top sheet is configured to wick moisture into the filler away from the user, but not out of the filler toward the user.

The primary goal of the pad is to remove moisture away from the body so that the user has a dry layer of fabric to sleep on, thereby allowing the user to sleep in comfort. The protection of the mattress, mattress pad and sheets are secondary benefits. This is just the opposite of a prior art incontinence pad, the primary goal of which is to protect the mattress, mattress pad and sheets from moisture. Sleeping comfort of the user is only a secondary consideration in this case.

By way of example, and not by way of limitation, the top sheet may be a functional fabric woven from fibers made of a composite of polyester, ceramic and bamboo. The top sheet 202 may have a thread count of such fibers between about 200 and about 400 threads per inch. One commercially available example of such a functional fabric is sold under the name Trek-Dry by Tiong Liong Corporation of Taichung Taiwan, R.O.C. A thickness of the top sheet 202 may be, e.g., about 1 mm.

By way of example, and not by way of limitation, the moisture absorbent filler 204 can be layer moisture absorbent cloth, e.g., made of 100% antimicrobial polyester. An example of an antimicrobial polyester material is an antimicrobial polyester staple fiber sold by Woongjin Chemical Co. Ltd of Seoul, Korea. Alternatively, the absorbent filler 204 could be a combination of a Cotton/Polyester or a Bamboo/Polyester mix. A thickness of the moisture absorbent filler 204 may be between about 3 millimeters and about 10 millimeters, e.g., about 5 millimeters.

The bottom layer 206 can be made of cloth, such as 100% cotton that is not moisture-proof from the outside. As used herein the term “not moisture-proof from the outside” refers to a material is configured such that it does not act as a barrier to transport of moisture from the outside of the bottom layer 206 to the filler 204. In some embodiments, the bottom layer 206 may be made of a material that is moisture-proof from the inside, e.g., a technical fabric that is configured acts as a barrier to transport of moisture from the filler 204 to the outside of the bottom layer 206.

The bottom layer 206 may be made of a material that is friction enhanced, e.g., by rubberizing at least a portion the outside surface 207 of the bottom layer 206. Such rubberization can enhance friction between the bottom layer 206 and a bed sheet, thereby increasing comfort to the user since the pad would tend to stay flatter.

The construction of the pad 104 provides several advantages over whereas an incontinence pad may be design to retain up to 36 oz of fluid and can be concentrated in a small area, the sweat pad 104 is designed to trap a much smaller volume of fluid (e.g., up to 6 oz) and it is spread over a much larger surface. Therefore the bottom layer 206 need not be a moisture barrier that acts as a barrier to moisture from the outside to the filler 204, as the absorbent filler can hold such a relatively small amount of fluid. Furthermore, because the bottom layer 206 is not waterproof from the outside, during regular laundering, the pad 104 can be washed and dried like a regular terrycloth towel.

The top and bottom sheets 202 and 206 preferably have dimensions that are large enough to cover the body of a typical user. The moisture absorbent filler sheet 204 has smaller dimension than the top and bottom sheets 202 and 206 and can be fixed between the top and bottom sheets 202 and 206. By way of example, the filler sheet 204 may be fixed between the top and bottom sheets 202, 206 by sandwiching the three sheets and sewing them together. Alternatively, the filler sheet 204 may be removably fixed between the top and bottom sheets 202, 206. For example, the top and bottom sheets may be sewn together along one edge and releasably fastened along the other three edges, e.g., by a zipper or a hook and loop fastener, such as Velcro. The filler sheet may optionally be releasably secured to either top or bottom sheet or both, e.g., by hook and loop fasteners.

Referring again to FIG. 1A and FIG. 2, the user 106 can use the sweat pad 104 by placing it on the bed and pillow 102 and lying on it. As seen in FIG. 3, when the user sweats, the top sheet 202 wicks moisture 201 away from the user's skin and into the filler 204 keeping the user's skin dry. The bottom layer 206 provides sufficient friction with respect to an underlying sheet to enhance the comfort of the user.

In an alternative embodiment depicted in FIG. 1B, the sweat pad 104 may include a pocket formed from an additional piece of cloth 208. The pocket or sleeve can be sown onto the bottom layer 206 proximate an end thereof to receive a pillow 103. The additional piece of cloth 208 may be made of the same type of material as the bottom layer, e.g., cotton. The additional piece of cloth 208 may be sewn as a pocket, e.g., with one end open to receive the pillow. Alternatively, the additional piece of cloth 208 may be sewn as a sleeve with two opposite ends open to receive the pillow 103.

In another alternative embodiment, illustrated in FIG. 1C, the night sweat pad 104 may be configured as a pillow case 105, e.g., by folding a composite material like that shown in FIG. 2 double and sewing a seam 107 along two adjacent edges to form a pocket to receive a pillow 103. In use, the technical fabric of the top sheet 202 may be on the outside of the resulting pillow case so that a user's head and/or neck can rest against the technical fabric layer.

As may be seen from the foregoing discussion, embodiments of the present invention provide a night sweat pad that can wick moisture in the form of sweat away from a user keeping the user dry while providing a comfortable surface on which to sleep. Although the construction of a night sweat pad according to embodiments of the present invention might at first appear similar to constructions used for nighttime clothing, outdoor apparel, footwear, incontinence pads or sanitary napkins, the present construction is different in a several counterintuitive ways.

Constructions used for nighttime apparel typically only contain a technical fabric that wicks moisture away from the body to the air and, therefore, lack a middle filler or outer cloth layer, which would undesirably trap moisture and add to the weight of an article of nighttime clothing. Constructions used for outdoor apparel are specifically designed to keep moisture out of the filler in order to prevent the apparel from becoming heavy when exposed to moisture. The present construction actually wicks moisture into the filler where it can be prevented from entering the bedding by the regular cloth back layer. Thus, the present construction would be quite undesirable in an article of nighttime or outdoor clothing that is worn by a user.

Furthermore, the present construction would be extremely unsuitable for use in incontinence pads or sanitary napkins since the back sheet is made of cloth, as opposed to a moisture-impermeable material. Moisture in the form of blood or urine and odor could undesirably leak out through a cloth back layer and soil the bedding. The use of cloth would also add unnecessary expense to a disposable incontinence pad or sanitary napkin.

However, the present construction can be quite advantageous when used as a night sweat pad placed between a user and the user's bedding because the user's skin and the bedding can be kept dry while in use. The cloth back sheet retains moisture due to sweat and allows the night sweat pad to be laundered and dried out in a conventional manner and reused.

The reader's attention is directed to all papers and documents which are filed concurrently with this specification and which are open to public inspection with this specification, and the contents of all such papers and documents incorporated herein by reference.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

Cotton is advantageous for a night sweat pad because you get more friction with cotton than a more slippery material such as polyester. The friction is desirable for getting the night sweat pad to stick to a bedsheet in a comfortable manner. In addition, non waterproof material, such as cotton is more readily available and less expensive than polyester or nylon.

The construction of the pad 104 is distinguishable from a prior art material used for outdoor wear or footwear. The prior art material is similar in construction in that the technical fabric is worn closest to the skin and wicks moisture away. In embodiments of the present invention, by contrast, the outermost layer is water permeable from the outside instead of being water proof from the outside. However, the outer layer of a jacket or footwear would not be made of cotton that does not act as a barrier to moisture from the outside. Such a material would be too moisture absorbent for outdoor wear or footwear.

While the above is a complete description of the preferred embodiment of the present invention, it is possible to use various alternatives, modifications and equivalents. Therefore, the scope of the present invention should be determined not with reference to the above description but should, instead, be determined with reference to the appended claims, along with their full scope of equivalents. Any feature, whether preferred or not, may be combined with any other feature, whether preferred or not. In the claims that follow, the indefinite article “A”, or “An” refers to a quantity of one or more of the item following the article, except where expressly stated otherwise. The appended claims are not to be interpreted as including means-plus-function limitations, unless such a limitation is explicitly recited in a given claim using the phrase “means for.”

What is claimed is:

1. A reusable sweat pad designed to be placed on a bed and/or pillow and under a user for wick away the moisture from a user comprising:
a moisture absorbent filler sandwiched between a top moisture-permeable sheet and a bottom cloth sheet, wherein the top moisture-permeable sheet is configured to wick moisture away from the user and toward the moisture absorbent filler, wherein the bottom cloth sheet is made of a material that is not moisture-proof from the outside.

2. The sweat pad of claim 1 wherein the top moisture-permeable sheet is configured to prevent moisture from being wicked from the filler toward the user.

3. The sweat pad of claim 1, wherein the top moisture-permeable sheet comprises a technical fabric woven from composite fibers made of bamboo, ceramic and polyester.

4. The sweat pad of claim 1, wherein the top moisture-permeable sheet has a thickness of about 1 mm and about.

5. The sweat pad of claim 1, wherein the moisture absorbent filler sheet comprises 100% polyester.

6. The sweat pad of claim 5, wherein the moisture absorbent filler sheet comprises of 100% antimicrobial polyester.

7. The sweat pad of claim 1, wherein the moisture absorbent filler sheet has a thickness between about 3 mm and about 10 mm.

8. The sweat pad of claim 1, wherein the bottom cloth sheet comprises a 100% cotton.

9. The sweat pad of claim 1, wherein the bottom cloth sheet comprises a material that is moisture-proof from the inside.

10. The sweat pad of claim 1, wherein the bottom cloth sheet has a thickness of about 1 mm.

11. The sweat pad of claim 1, wherein the moisture absorbent filler sheet is releasably secured between the top moisture-permeable sheet and the bottom sheet.

12. The sweat pad of claim 1, wherein the sweat pad is characterized by a length between about 34 inches and about 75 inches and a width between about 21 inches and about 39 inches.

13. The sweat pad of claim 1 wherein the bottom cloth sheet includes a friction-enhanced outer surface.

14. The sweat pad of claim 13, wherein the friction-enhanced outer surface includes a portion that has been rubberized.

15. The sweat pad of claim 1, wherein the sweat pad is configured as a pillow case.

16. A reusable sweat pad designed to be placed on a bed and/or pillow and under a user for wick away the moisture from a user comprising:
a moisture absorbent filler sandwiched between a top moisture-permeable sheet and a bottom cloth sheet, wherein the top moisture-permeable sheet is configured to wick moisture away from the user and toward the moisture absorbent filler, wherein the bottom cloth sheet wherein the bottom cloth sheet includes a friction-enhanced outer surface.
